(19) United States
(12) Patent Application Publication
(10) Pub. No.: US 2012/0277582 A1
(43) Pub. Date: Nov. 1, 2012

(54) DEVICES AND METHODS FOR OSTEOLYTIC LESION ASSESSMENT USING A STEERABLE CATHETER

(75) Inventor: Masoumeh Mafi, Mountain View, CA (US)

(73) Assignee: KYPHON SARL, Neuchatel (CH)

(21) Appl. No.: 13/094,451

(22) Filed: Apr. 26, 2011

(51) Int. Cl. A61B 5/103; A61B 6/00

(52) U.S. Cl. 600/431; 600/587

(57) ABSTRACT

A method of assessing the volume of a lesion in a bone comprises inserting a steerable catheter comprising an expandable structure, a suction member, and a steerable element into the bone along a longitudinal axis. The method further comprises steering the steerable element away from the longitudinal axis toward the lesion, removing cellular matter from the lesion using the suction member, and inflating the expandable structure with inflation medium to create a cavity defining the boundary of the lesion. The method also comprises measuring the volume of inflation medium in the expandable structure, thereby determining the volume of the cavity.

### BACKGROUND OF THE INVENTION

Bone loss is commonly associated with several diseases, including osteolysis, metastatic lesions, and osteoporosis. Though bone loss often refers to the dissolution of bone secondary to a variety of medical conditions, the term osteolysis generally refers to a bonc resorption problem common to artificial joint replacements such as hip replacements, knee replacements, and shoulder replacements. Osteolysis often occurs in the bone adjacent to an orthopedic implant, such as a hip or knee implant. As the body attempts to clean the orthopedic implant wear particles from the surrounding bone, an autoimmune reaction may be triggered. This autoimmune reaction causes the resorption of living bone tissue in addition to resorption of the wear particles. This bone resorption forms voids or osteolytic lesions in the bone. Osteolytic lesions are typically soft and spongy, and are unsupportive of orthopedic implants. An osteolytic lesion can cause a well-fixed implant to loosen. To treat osteolysis in the area of an implant, it is often necessary to conduct a revision surgery in which the old implant is removed, the lesion is debrided, and a larger revision implant is inserted.

In addition to osteolytic lesions secondary to implant reactions, another common form of osteolytic lesions are “punched out” osteolytic lesions secondary to metastatic cancer. “Punched-out” osteolytic lesions are characteristic of metastatic lung and breast cancers and multiple myeloma.

Both types of osteolytic lesions can trigger a host of serious medical problems in patients, including severe pain, bone fractures, life-threatening electrolyte imbalances, and nerve compression syndromes. One of the treatments for alleviating the symptoms of osteolytic lesions involves clearing the lesion of cellular debris and filling it with biomaterial or bone cement. Because patients with osteolytic lesions are typically older, and often suffer from various other significant health complications, many of these individuals are unable to tolerate invasive surgery. Therefore, in an effort to more effectively and directly treat osteolytic lesions, minimally invasive procedures may be utilized to repair the bone by assessing the volume and location of the lesion and then injecting an appropriate amount of flowable reinforcing material into the osteolytic lesion. Shortly after injection, the filling material hardens, thereby filling the lesion and supporting the bone internally.

In contrast to an open procedure for the same purpose, a minimally invasive, percutaneous procedure will generally be less traumatic to the patient and result in a reduced recovery period. However, minimally invasive procedures present numerous challenges. For example, proper assessment of the size and location of the osteolytic lesion is essential to the accurate location of the lesion and precise delivery of the appropriate amount of reinforcing material within the lesion. Without direct visual feedback into the operative location, accurately selecting, sizing, placing, and/or applying minimally invasive surgical instruments and/or treatment materials/devices can be difficult.

Accordingly, there exists a need for instrumentation and techniques that facilitate the more effective and efficient treatment of bone dissolution using minimally invasive procedures. Therefore, it would be advantageous to provide a system and method of assessing and repairing areas of bone dissolution, including osteolytic lesions, using minimally invasive instrumentation and techniques.

### SUMMARY OF THE INVENTION

The present invention relates to devices and methods to facilitate minimally invasive assessment of the location and volume of bone lesions, including osteolytic lesions and other areas of bone loss.

One embodiment of the invention provides a method of assessing the location and volume of a lesion in a bone that comprises inserting a steerable catheter comprising an expandable structure, a suction member, and a steerable element into the bone along a longitudinal axis. The method further comprises steering the steerable element away from the longitudinal axis toward the lesion, removing cellular matter from the lesion using the suction member, and inflating the expandable structure with inflation medium to create a cavity defining the boundary of the lesion. The method also comprises measuring the volume of inflation medium in the expandable structure, thereby determining the volume of the cavity.

Another embodiment of the invention provides a method of assessing the volume of a lesion in a bone that comprises inserting a steerable catheter comprising an expandable structure, a suction member, and a steerable element into the bone. In this embodiment the steerable element includes integrated radiopaque markers. The method further includes the steps of steering the steerable element away from the longitudinal axis toward the lesion, removing cellular matter from the lesion using the suction member, and inflating the expandable structure with inflation medium to create a cavity defining the boundary of the lesion. The method also comprises imaging the cavity while the expandable structure is inflated, visualizing the radiopaque markers in the imaged cavity, and measuring the volume of inflation medium in the expandable structure, thereby determining the volume of the cavity.

Yet another embodiment of the present invention provides a method of assessing the volume of a lesion in a bone that comprises inserting a steerable catheter comprising an expandable structure, a suction member, and a steerable element into the bone. In this embodiment, the steerable element is connected to the expandable structure. The method further includes the steps of steering the steerable element away from the longitudinal axis toward the lesion, removing cellular matter from the lesion using the suction member, inflating the expandable structure with inflation medium to create a cavity defining the boundary of the lesion, and articulating the steerable element to change a configuration of the expandable structure. The method also comprises imaging the cavity while the expandable structure is inflated and measuring the volume of inflation medium in the expandable structure, thereby determining the volume of the lesion.

In some embodiments of the present invention, the steerable catheter further comprises a controller that steers the steerable element.

In some embodiments of the present invention, the method further comprises filling the cavity with a material that sets to a hardened condition.

Further aspects, forms, embodiments, objects, features, benefits, and advantages of the present invention shall become apparent from the detailed drawings and descriptions provided herein.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIGS. 1a and 1b are cross-sectional side views of a first embodiment of a steerable catheter with an expanded structure surrounding the tip of the steerable element. FIG. 1a shows the steerable catheter when the steerable element is straight. FIG. 1b shows the steerable catheter when the steerable element is curved.

FIGS. 2a and 2b are cross-sectional side views of an exemplary steering element for use in a steerable catheter. FIG. 2a shows the steerable element when the steerable element is straight. FIG. 2b shows the steerable element when the steerable element is curved.

FIG. 3 is a cross-sectional side view of a first embodiment of the steerable catheter inserted into a bone lesion in the ilium.

FIG. 4 is a cross-sectional side view of a first embodiment of the steerable catheter aspirating material from a bone lesion in the ilium.

FIG. 5 is a cross-sectional side view of a first embodiment of the steerable catheter expanding an expandable structure within a bone lesion in the ilium.

FIG. 6 is a perspective view of an embodiment of the expandable structure having heating elements.

FIG. 7 is a cross-sectional side view of a first embodiment of the steerable catheter filling an expandable structure with inflation medium.

FIG. 8 is a cross-sectional side view of a first embodiment of the steerable catheter deflating an expandable structure.

FIG. 9 is a cross-sectional side view of a first embodiment of the steerable catheter filling a bone lesion within the ilium with bone filler material.

FIG. 10 is a cross-sectional side view of an expandable structure filled with bone filler material left within a bone lesion within the ilium.

FIG. 11 is a cross-sectional view of a second embodiment of a steerable catheter with an expanded structure surrounding the tip of the steerable element.

FIG. 12 is a cross-sectional view of a third embodiment of a catheter with an expanded structure surrounding the tip of the catheter.

### DETAILED DESCRIPTION OF THE PREFERRED EMBODIMENTS

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

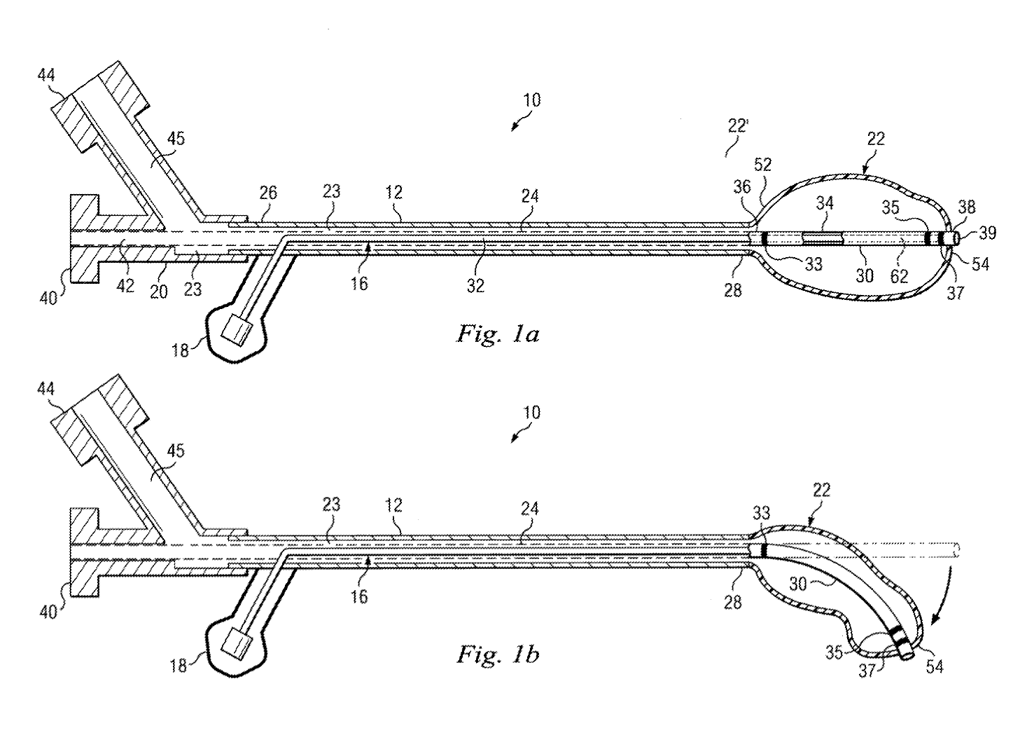

FIG. 1 a shows a cross-section of an embodiment of a steerable catheter 10 that can be used in a surgical procedure, such as the assessment and repair of an osteolytic lesion. The steerable catheter 10 includes a shaft 12, a steering mechanism 16, a controller 18 for controlling the steering mechanism 16, a connector 20, and an expandable structure 22.

The shaft 12 is an elongate, hollow, cylindrical tube defining a lumen 24. The shaft 12 includes a proximal end 26 and a distal end 28. The proximal end 26 is relatively rigid, having sufficient column strength to push through cancellous bone. The shaft carries a lumen 23 and a lumen 24, which is partially disposed within lumen 23 and houses the steering mechanism 16. In other embodiments, the shaft 12 may include greater than two lumens. The lumen 23 is configured to carry flowable material, including but not limited to inflation media. The lumen 24 is configured to carry flowable material, including but not limited to cellular matter and flowable bone cement (prior to hardening). In some embodiments, the lumen 24 could accept a stiffening stylet or guidewire (not shown for simplicity), with the steering mechanism 16 either sharing the space within the lumen 24 or being positioned alongside of the lumen 24.

The steering mechanism 16 includes a steerable element 30 and a shaft portion 32. FIG. 1a shows the steerable catheter when the steerable element 30 is straight. FIG. 1b shows the steerable catheter when the steerable element 30 is curved. The shaft portion 32 resides within the lumen 24 of the shaft 12 and couples the steerable element 30 to the controller 18. The steerable element 30 is a cylindrical tube defining a lumen 34 that is in connection with the lumen 24. In other embodiments, the steerable element 30 can have multiple lumens. In some embodiments, the multiple lumens of the steerable element 30 are contiguous with the multiple lumens of the shaft 12. In some embodiments, the lumen 34 of the steerable element 30 is lined by a continuous, flexible, nonporous membranous tube, having a distal end attached to the distal end 38 of the steerable element 30 and a proximal end attached to the proximal end 36 of the steerable element 30 such that the lumen of the tube and the lumen 24 are contiguous. In some embodiments, the steerable element 30 has a lower durometer value than the shaft 12, thereby facilitating deflection of the steerable element against the walls of a bone lesion, for example.

The steerable element 30 includes a proximal end 36 and a distal end 38. The distal end 38 can be rounded or beveled to facilitate passage through bone, or can be flattened to minimize unnecessary movement through or trauma to surrounding tissues. The distal end 38 can include an opening 39 in communication with the lumen 34 to permit the expression from and entry of material into the steerable catheter 10. The opening 39 may be provided on a distally facing surface, on a laterally facing surface, or on an inclined surface of the distal end 38 of the steerable element 30.

The steerable element 30 includes radiopaque markers 33 and 35 disposed near the proximal end 36 and the distal end 38, respectively. In this embodiment, the radiopaque markers 33, 35 are circular bands extending circumferentially around the steerable element 30. In other embodiments, the radiopaque markers may be configured in a variety of shapes and sizes and may be positioned in variety of locations along the steerable element 30. In some embodiments, the steerable element 30 may include any number of radiopaque markers. In other embodiments, the steerable element 30 may not include any radiopaque markers.

The steerable element 30 includes an anchoring element 37 disposed immediately proximal to the distal end 38. The anchoring element 37 illustrated in FIG. 1a is a circular band extending circumferentially around the steerable element 30. In other embodiments, the anchoring element 37 may be shaped and configured in a variety of shapes and sizes, provided that the anchoring element is located near the distal end 38. The anchoring element 37 may be connected to the steerable element 30 by a variety of methods, including welding, soldering, and adhering with adhesive. In some embodiments, the anchoring element 37 may be integrally attached to the steerable element 30.

In some embodiments, the length X of the steerable element 30 is at least about 10% of the length Y of the shaft 12, and in other embodiments at least about 15%, 25%, 35%, or more of the length Y of the shaft 12 for optimal operation of the steerable catheter 10. One of ordinary skill in the art will realize that the ratio of the lengths X:Y can vary depending upon the desired functionality and clinical application of the steerable catheter 10.

The controller 18 permits the physician to control the movement of the steerable element 30 via the steering mechanism 16. The controller 18 is coupled to the proximal end 26 of the shaft 12. The controller 18 is shaped and configured to interface with the shaft portion 32 of the steering mechanism 16 such that when the physician maneuvers the controller 18, the shaft portion 32 of the steering mechanism 16 moves the steerable element 30 in a plurality of directions. The controller 18 may be any one of, for example, a rotatable wheel, a trigger mechanism, or a plurality of push-buttons. The steerable element 30 is “actively” steerable because it can be configured into a variety of shapes (i.e., articulated) by the controller 18 without any other external forces acting on the steerable element 30 (in contrast to a passive structure like a bent shape-memory wire that can only be straightened by application of external forces like a sheath).

The connector 20 is coupled to a proximal end 26 of the shaft 12. The connector 20 may be configured as a Luer lock connector, but can also be configured in a wide variety of other connector options. For example, the connector 20 may be configured as a hose barb or slip fit connector. The connector 20 includes a port 40 for withdrawing cellular material from the lesion through the lumen 34 of the steerable element 30 and for delivering flowable bone cement into the lesion. The port 40 provides for the releasable connection of the steerable catheter 10 to a source of flowable material. The lumen 42 of the port 40 is fluidly connected to the lumen 24 of the shaft 12 such that material can flow from a source, through the port 40 into the port lumen 42, into the lumen 24 of the shaft 12, and out the distal opening 39 of the steerable element 30.

The connector 20 includes a port 44 for receiving inflation material (e.g., saline solution or contrast solution) for inflating the expandable structure 22. The port 44 provides for the releasable connection of the steerable catheter 10 to a source of inflation material. The lumen 45 of the port 44 is fluidly connected to the lumen 23 of the shaft 12 such that material can flow from a source, through the port 44 into the port lumen 45, into the lumen 23 of the shaft 12, and out the distal end 28 of the shaft 12 into the lumen of the expandable structure 22.

Note that in various embodiments, the connector 20 can include any number of ports 40. In some embodiments, a plurality of ports 40 are present, for example, for irrigation, suction, inflation, introduction of medication or flowable bone cement, or as a port for the introduction of other tools, such as a light source, cautery, cutting tool, visualization devices, or the like. In a single embodiment of the steerable catheter 10, the connector 20 can include one port for inflation of the expandable structure 22, one port for the suction of the contents of the bone lesion, and one port for delivery of the bone cement into the bone lesion.

The expandable structure 22 includes a proximal portion 52 and a distal portion 54. In the pictured embodiment, the proximal portion 52 of the expandable structure 22 is attached to the distal end 28 of the shaft 12, but in other embodiments, the expandable structure 22 need not be attached to the shaft or may be attached to a different portion of the shaft 12. For example, an unattached expandable structure 22 can be inserted through the connector port 40, advanced through the lumen 24 of the shaft 12 into the lumen 34 of the steerable element, and emerge from the distal opening 39 of the steerable element 30. In other embodiments, the expandable structure 22 may be secured inside or outside the shaft 12.

In the embodiment pictured in FIGS. 1a and 1b, the expandable structure 22 surrounds the steerable element 30 and is connected to the anchoring element 37 at the distal end 38 of the steerable element 30. The expandable structure 22 is disposed around the entire length of the steerable element 30. The anchoring element 37 of the steerable element 30 is coupled to the distal portion 54 of the expandable structure 22. In other embodiments, the distal end 54 of the expandable structure 22 may attach to other locations on the steerable element 30 or may not attach to the steerable element 30. For example, in some embodiments the expandable structure 22 may completely encompass the steerable element 30, with the distal end 38 of the steerable element 30 completely unattached and disposed within the lumen of the expandable structure 22. By incorporating an actively steerable element into the expandable structure 22, repositioning of the expandable structure 22 can be beneficially performed after the expandable structure 22 has been inserted into the target surgical location.

Although the embodiment pictured in FIG. 1a includes a single-chamber, teardrop-shaped expandable structure 22, the expandable structure 22 can have any shape or construction such that it may be contained within or carried by the steerable catheter 10 (for example, a spherical balloon, a multi-chambered balloon, or a balloon with internal or external reinforcing features).

The expandable structure 22 can be comprised of a flexible and biocompatible material common in medical device applications, including, but not limited to, plastics, polyethelene, mylar, rubber, nylon, polyurethane, latex, metals, or composite materials. For example, the expandable structure 22 can be formed from a compliant (e.g., latex), semi-compliant (e.g., polyurethane), or non-compliant (e.g., nylon) material. The connector 20, the shaft 12, and the steering mechanism 16 are comprised of biocompatible materials that are more resistant to expansion than the material of the expandable structure 22, including, but not limited to, metals such as stainless steel, ceramics, composite material, or rigid plastics. In alternate embodiments, similar materials for the expandable structure 22, the connector 20, the shaft 12, and the steering mechanism 16 may be used, but in different thicknesses and/or amounts, thereby crafting the expandable structure 22 to be more prone to expansion than the remaining components of the steerable catheter 10.

It is important to note that minimally invasive procedures such as the one described herein are typically performed under fluoroscopy or other imaging modalities to allow the physician to visually observe and monitor the surgical activity within the patient. Therefore, in some embodiments, radiopaque markers can be placed at various locations on the steerable catheter 10 to facilitate appropriate placement of the steerable element 30 within the lesion. In various other embodiments, the steerable element 30 can be formed from, or can include, radiopaque materials. In other embodiments, the steerable catheter 10 can include visible indicia such as, for example, a marker visible via other imaging modalities such as ultrasound, CT, or MRI.

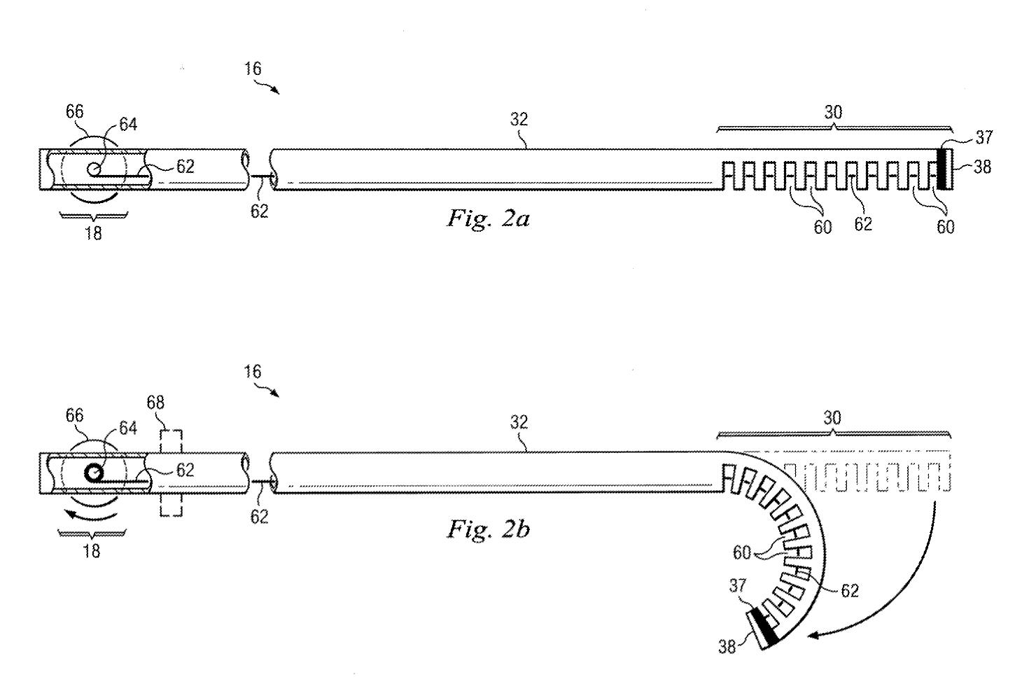

A variety of controllers 18 may be used with the steerable catheter 10, for actuating the curvature of the steerable element 30. Preferably, the controller 18 allows for one-handed operation of the steerable catheter 10 by a physician. FIGS. 2a and 2b show an exemplary embodiment of the controller 18 and the steering mechanism 16, in which the controller 18 is operated by a rotatable member, a thumbwheel 66. A plurality of slots 60 extend partially circumferentially around part of the steerable element 30, providing a plurality of flexion joints to facilitate bending. An axially movable cable 62, having a proximal end and a distal end, is attached at its distal end to anchoring element 37 at the distal end 38 of the steerable element 30 and runs through the shaft 32 to the controller 18. In some embodiments, the cable 62 is coated with a nonstick material such as Teflon. Alternatively or additionally, in some embodiments the cable 62 is isolated and surrounded by flexible tubing, for example polyimide tubing. The cable 62 can be attached to the anchoring element 37 by an adhesive, welding, soldering, crimping, or the like. In this embodiment, the controller 18 includes a spindle 64 mounted on the thumbwheel 66, with the cable 62 coupled at its proximal end to the spindle 64 such that the cable 62 is axially translatable.

FIG. 2b illustrates how rotating the thumbwheel 66 winds the cable 62 around the spindle 64, thereby causing the slotted steerable element 30 to curl away from the longitudinal axis of the shaft 32. The controller 18 is configured to provide an axial pulling force in the proximal direction toward the proximal end of the cable 62. This in turn exerts a proximal pulling traction on the anchoring element 37 of the steerable element 30, which is attached to the distal end of the cable 62. When the thumbwheel 66 is rotated in a first direction, a proximally directed tension force is exerted on the cable 62. actively changing the curvature of the steerable element 30 as desired. The slots 60 determine the direction of curvature for the steerable element 30. The slotted side shortens under compression, while the opposite side of the steerable element 30 retains its axial length, causing the steerable element 30 to curl in the direction of the slotted side of the steerable element 30. The degree of deflection can be observed fluoroscopically, and/or by other printed or other indicia associated with the controller 18.

In this embodiment, the plurality of slots 60 are preferably occluded, to prevent materials such as cellular matter or flowable bone cement from escaping through the slots 60. Occlusion of the slots 60 may be accomplished in a variety of ways, such as by positioning a thin, flexible, membranous tube coaxially about the exterior surface of the steerable element 30 and securing the tube across the slots 60. In other embodiments, the material travelling through the lumen 34 of the steerable element 30 may be prevented from escaping through the plurality of slots 60 by the provision of a thin, flexible, membranous tube carried on the interior surface defining the lumen 34 of the steerable element 30 (thereby physically separating the interior of the lumen 34 from the slots 60).

In one embodiment, the shaft 32 includes features 68 (e.g., flanges, a collar, ribs, or extensions, among others) that facilitate rotation of the steering mechanism 16. In various other embodiments, such features can be positioned elsewhere on the steerable catheter 10.

In some embodiments, the shaft 32 can be formed from a shape-memory material (e.g., Nitinol) such that once the cable 62 is allowed to unspool from the spindle 64 (e.g., by releasing or unlocking the thumbwheel 66), the steerable element 30 returns to its original, straight configuration. In other embodiments, the cable 62 can be selected to have sufficient rigidity to “pull” the steerable element 30 back into a straight configuration. In other embodiments, the steering mechanism 16 can include multiple cables to control the configuration of the steerable element 30. For example, in one embodiment, the steering mechanism 16 can include a second cable in opposition to the cable 62 to flex the steerable element 30 back into a straight configuration (or to curve the steerable element 30 in an entirely different direction).

It is important to note that although FIGS. 2a and 2b depict the steering mechanism 16 as having a slotted steerable element 30 for exemplary purposes, the steering mechanism 16 and the steerable element 30 can have any construction that provides active steering capability to the steerable catheter 10. Alternative controllers 18 include rotatable knobs, slider switches, pull tabs, linear actuators, compression grips, triggers such as on a gun grip handle, or others depending upon the desired functionality of the steerable catheter 10. In addition, in various embodiments, the steerable element 30 could include a flexible sleeve over a flexible internal member between parallel control cables, such that each cable pulls the flexible member in a different direction. In various other embodiments, steerable element 30 could include a coil of wire surrounding a relatively rigid core that pushes distally to flex the coil.

In some embodiments, the controller 18 allows for continuous adjustment of the curvature of the steerable element 30 throughout a working range. In other embodiments, the controller 18 is configured for discontinuous or step-wise adjustment of the curvature of the steerable element 30, e.g. via a racheting mechanism, preset slots, deflecting stops, a rack an pinion system with stops, a racheting band (an adjustable zip-tie), an adjustable cam, or a rotating dial of spring-loaded stops. In yet other embodiments, the controller 18 may include an automated mechanism, such as a motor or a hydraulic system, to facilitate adjustment. Various other embodiments of the controller 18 will be readily apparent to one of skill in the art.

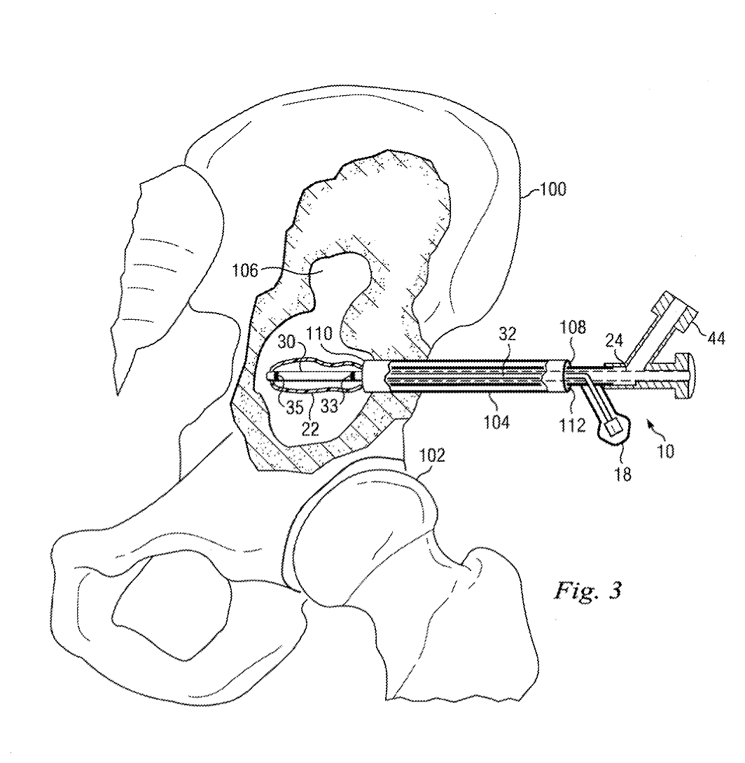

FIGS. 3-5, 7, and 8 show an exemplary bone lesion assessment procedure using a steerable catheter 10 that incorporates an actively steerable element 30 (as described with respect to FIGS. 1a-1b). It is important to note that while the use of a single steerable catheter is depicted for exemplary purposes, in various other embodiments any number of steerable catheters 10 can be used. In some embodiments, the actively steerable catheter 10 can be used with conventional (i.e., not actively steerable) catheters, including balloon catheters.

FIG. 3 is a cross-sectional view of a portion of a human hip, illustrating the acetabular joint connecting the ilium 100 and the head 102 of the femur. FIG. 3 illustrates the steerable catheter 10 positioned within a cannula 104 inserted into the ilium 100, with the steerable element 30 positioned inside a bone lesion 106 demonstrating a multi-loculated area of bone loss. In the pictured embodiment of the present invention, an exemplary surgical method comprises inserting the cannula 104 percutaneously into a bone, such as the ilium 100. The cannula 104 may be any type and size of hollow insertion instrument. In FIG. 3, the cannula 104 is an elongate, hollow, cylindrical tube having a proximal end 108, a distal end 110, and a lumen 112. The cannula 104 is preferably comprised of a strong, non-reactive, and medical grade material such as surgical steel.

Typically, the cannula 104 would be docked into the exterior wall of the ilium 100 using a guide needle and/or dissector, after which a drill or other access tool (not shown) could be used to create a channel further into bone lesion. However, any method of cannula placement may be used. During insertion of the cannula 104, the positioning of the cannula 104 can be monitored using visualization equipment such as X-ray, CT, MRI scanning equipment, or any other surgical monitoring equipment commonly used by those of skill in the art. In the pictured embodiment, the distal end of the cannula 104 is positioned shallowly within the bone lesion 106, but the cannula 104 may be positioned anywhere within the bone lesion 106 in order to facilitate the minimally invasive assessment and repair procedure.

Once the cannula 104 is positioned within the bone lesion 106, the steerable catheter 10 can be positioned within the bone lesion. Under visual imaging monitoring, for example, fluoroscopic, CT, or MRI monitoring, the steerable catheter 10 can be inserted through the lumen 112 of the cannula 104 and advanced into the bone lesion 106, as shown in FIG. 3. The steerable element 30 is maintained in a straight configuration during advancement of the steerable catheter 10 through the cannula 104.

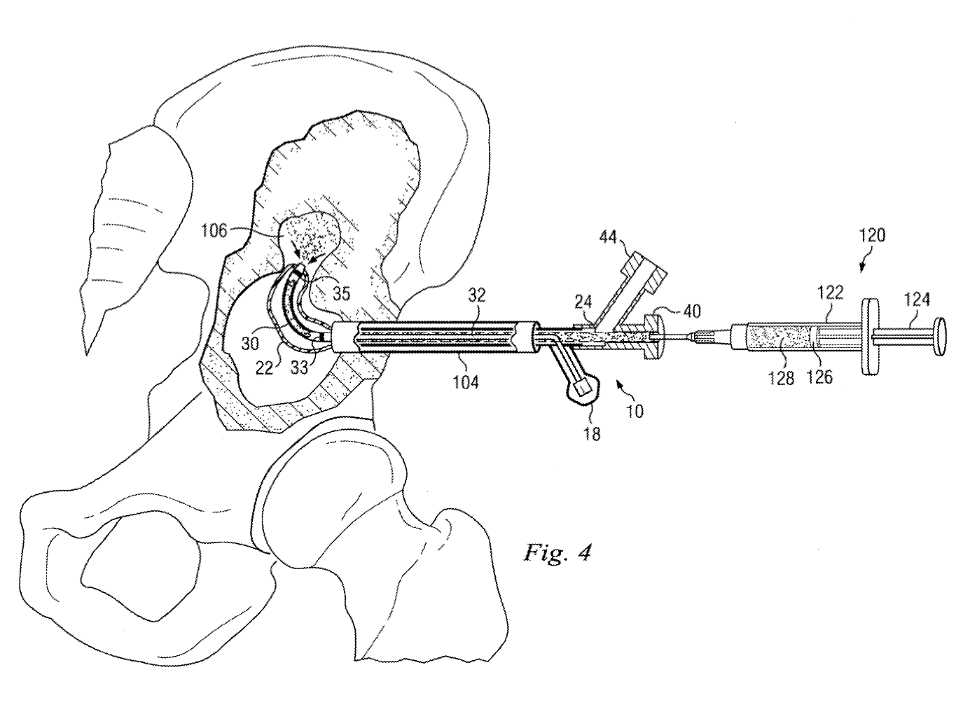

Then, as shown in FIG. 4, the controller 18 is manipulated to change the configuration of the steerable element 30. As illustrated by FIG. 4, the controller 18 can be manipulated to cause the steerable element 30 (via the shaft 32 of the steering mechanism 16) to curve upward and into a particular area of bone loss within the bone lesion 106. By imaging the radiopaque markers 33 and 35 located on the steerable element 30, the physician can visually localize and guide the steerable element 30 into a target location.

In some embodiments, a curette or other mechanical tool can be used to break up or scrape away portions of cancellous bone within the bone lesion 106 prior to the insertion of the steerable catheter 10. In this manner, the resistance encountered by the steerable element 30 as it moves within the bone lesion 106 can be minimized. However, besides providing greater positional control over the expandable structure 22, the active steering functionality of the steerable element 30 also provides significantly greater force generation capability than would be possible from passive shaping elements (e.g., a balloon catheter with a wire having a preformed bend within the balloon). Therefore, in some embodiments, the steerable catheter 10 itself can be used to scrape, cut, and/or compact the cancellous bone through the articulation of the steerable clement 30.

Once the distal end 38 of the steerable element 30 is positioned as desired within the bone lesion 106, a source of negative pressure such as a syringe assembly 120 (or any other container containing a fixed vacuum) can be removably attached to the port 40 such that the syringe assembly 120 can fluidly communicate with the lumen 24 of the shaft 12 of the steerable catheter 10. A mechanical pump or bulb or any other source of negative pressure could be used instead of a syringe assembly. The syringe assembly 120 comprises a syringe body 122 coupled to a syringe arm 124 holding a syringe plunger 126. Flowable material 128 can be loaded into and carried within the syringe body 122.

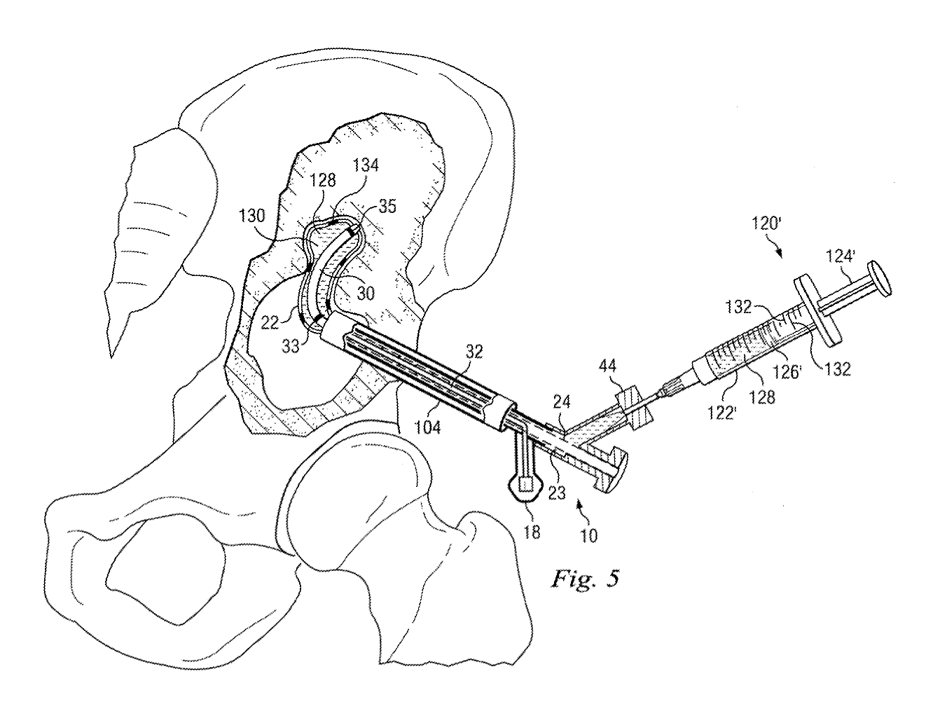

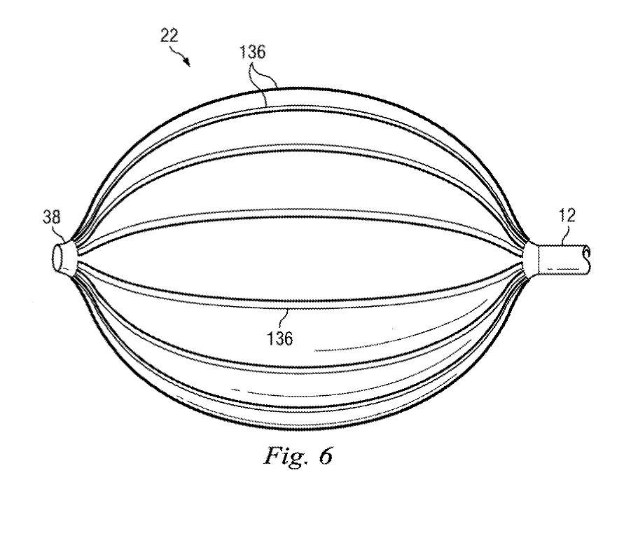

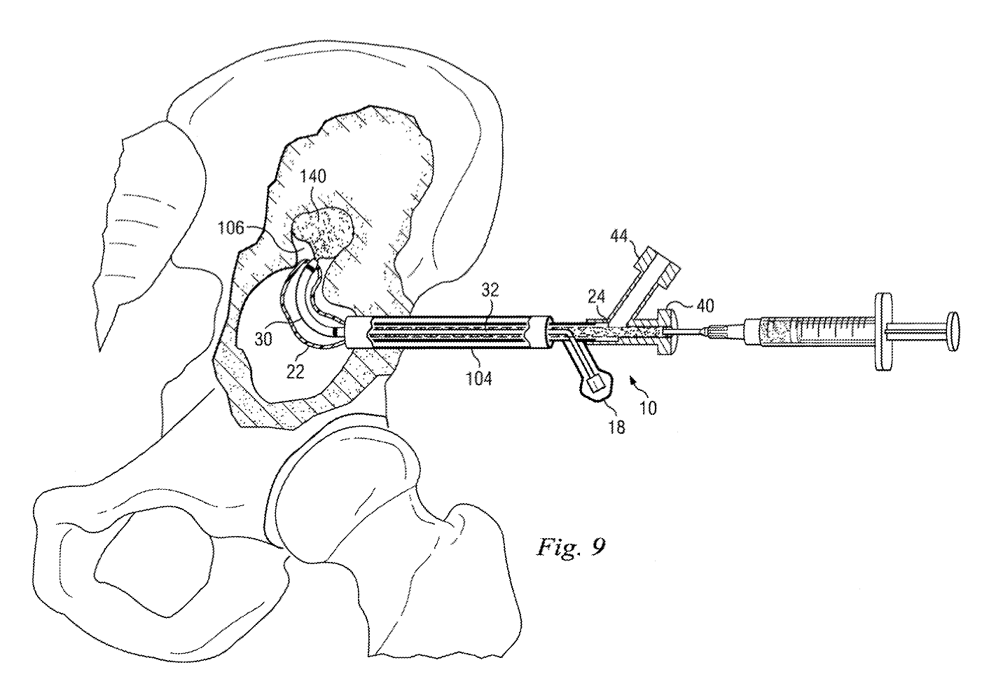

The syringe assembly 120 can be configured in any one of a variety of ways as is known in the art. Syringe bodies 122 possessing different lengths and/or different interior volumes can be provided to meet the particular delivery or suction objectives of the procedure. The syringe plunger 126 is positioned on a distal end of the syringe arm 124. The syringe plunger 126 desirably comprises a material, e.g., polyisoprene rubber, which creates a sealing engagement between the syringe plunger 126 and the interior wall of the syringe body 122 to create an expelling or suctioning force upon any flowable material 128 within the syringe body 122. The syringe arm 124 can axially move through the syringe body 122 either toward the port 40, thereby expelling the flowable material 128 from the syringe body 122 (as shown in FIGS. 5, 8, 10, and 11a), or away from the port 40, thereby aspirating or suctioning flowable material 128 into the syringe body 122 (as shown in FIGS. 4 and 9).

Once the syringe assembly 120 is removably attached to the port 40 such that the syringe plunger 126 is positioned within the syringe body 122 as close as possible to the port 40, the physician can begin suctioning or aspirating cellular matter out of the bone lesion 106. As the physician retracts the syringe arm 124, the syringe plunger 126 also moves proximally within the syringe body 122, thereby drawing material 128 through the lumen 34 of the steerable element 30, into the lumen 24 of the steerable catheter 10, and into the syringe body 122. If desired, the distal end 38 of the steerable element 30 can be advanced into or articulated around the bone lesion 106 more than once to ensure complete aspiration or suction of all the cellular matter within the bone lesion 106. The physician may reposition the cannula 104 within the bony lesion 106 and/or reposition the steerable catheter within the cannula 104 in order to optimally position the steerable element within the bony lesion 106. After the area inside the bone lesion 106 has been adequately suctioned to remove the extraneous matter from and create a hollow cavity within the bone lesion 106, the syringe assembly 120, containing the aspirated material 128, is removed from the port 40.

Once the bone lesion 106 has been suctioned and the distal end 38 of the steerable element 30 is positioned as desired within the bone lesion, the expandable structure 22 can be inflated as shown in FIG. 5 using a syringe assembly 120′ which may be substantially similar to the syringe assembly 120 except for the differences to be noted. The inflation can be performed with the use of a syringe assembly 120′ by injecting an inflation medium 128 (e.g., saline solution or contrast solution, among others) through the port 44 and the lumen 23 of the steerable catheter 10 into the lumen 130 of the expandable member 22. Injection of the inflation medium 128 is accomplished by depressing the syringe arm 124′, thereby advancing the syringe plunger 126′ toward the port 40 and expelling the inflation medium 128 into the steerable catheter 10 and the expandable structure 22.

The syringe assembly 120′ has volume indicia 132 displayed along the side of the syringe body 122′. The indicia 132 may run the entire length of the syringe body 122′ or just portions thereof as needed. Before injecting any inflation medium 128 into the steerable catheter 10, the physician can note the volume level of the inflation medium 128 contained within the syringe body 122′ by observing the position of the syringe plunger 126′ relative to the volume indicia 132. In the pictured embodiment, the physician injects the inflation medium 128 to expand the expandable structure 22 until the expandable structure 22 entirely occupies the bone lesion 106.

After expanding the expandable structure 22 to occupy the entire bone lesion 106, a physician may visually assess the location and size of the bone lesion 106 on an imaging modality such as X-ray, a CT, or an MRI equipment. If either a radiopaque inflation medium 128 is used or the expandable structure 22 contains radiopaque markers 134, this allows the physician to visualize the expandable structure 22 during inflation. The radiopaque markers 134 can be positioned on the exterior surface of the expandable structure 22 such that the physician can visualize the boundaries of the bone lesion 106 within the patient's body and estimate the volume of the bone lesion 106 based on the positional relationship of the radiopaque markers 134 and measuring their relative separation from each other. In different embodiments, the number and position of the radiopaque markers 134 can vary. The physician may also use the radiopaque markers 33, 35 of the steerable catheter 10 to aid in the volumetric assessment of the bone lesion 106.

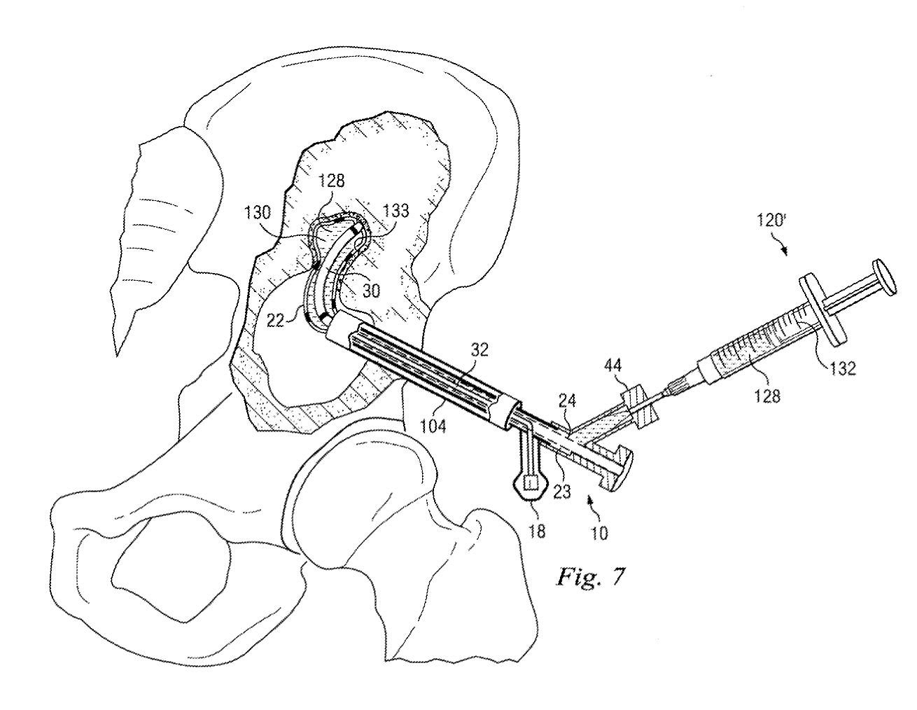

As shown in FIG. 7, in some embodiments, the physician may inject the inflation medium 128 to expand the expandable structure 22 until the expandable structure 22 compresses cancellous bone bordering the bone lesion 106. In this embodiment, as the expandable structure 22 is inflated, cancellous bone and cellular matter are displaced generally outward from the expandable structure 22 in a controlled manner, forming a compressed-bone region or “shell” 133 around a substantial portion of the outer periphery of the cavity. When the expandable structure 22 is deflated, a well-defined cavity with the surrounding “shell” 133 remains. The “shell” 133 can inhibit flowable bone cement from exiting the area of the bone lesion 106, thereby inhibiting extravasation of the bone cement and facilitating pressurization of the bone cement if needed.

FIG. 6 illustrates an embodiment of the expandable structure 22 including linear, flexible heating elements 136 positioned around the exterior surface of the expandable structure 22. The heating elements 136 function to help create and strengthen the “shell” 133, thereby preventing extravasation of flowable bone cement from the bone lesion 106.

As FIG. 7 indicates, after injecting the desired amount of inflation medium 128 into the expandable structure 22, the physician can calculate the amount of inflation medium 128 within the expandable structure 22. The volume of inflation medium 128 within the expandable structure 22 may be directly indicated by the indicia 132 or may be indirectly indicated by the indicia 132 displaying the amount of inflation medium 128 within the syringe body 122′ so that a certain reduction of inflation medium 128 from the syringe body 122′ indicates the amount of inflation medium 128 delivered to the expandable structure 22. In this instance, the physician can calculate the amount of inflation medium 128 within the expandable structure by, for example, subtracting the volume indicated by the final position of the syringe plunger 126′ (after injection) from the volume indicated by the initial position of the syringe plunger 126′ (before injection). In the alternative, the physician can calculate the amount of inflation medium 128 within the expandable structure 22 by summing the volumes of injected inflation medium 128 as indicated by the volume indicia 132. If the expandable structure 22 was expanded to occupy the entire bone lesion 106, then the amount of inflation medium 128 within the expandable device corresponds to the volume of the bone lesion 106.

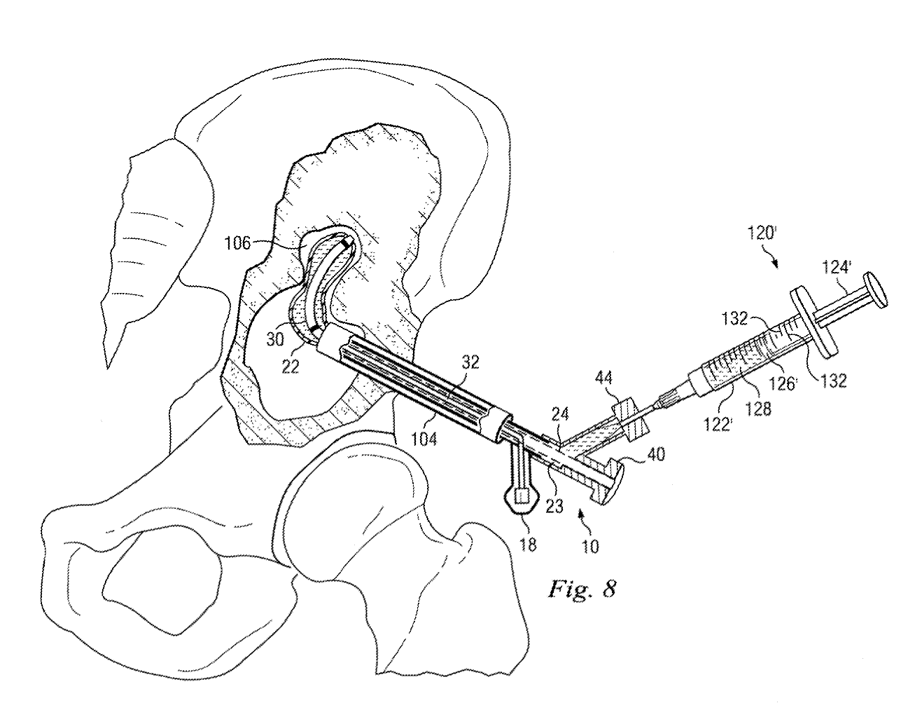

As FIG. 8 illustrates, the inflation medium 128 is withdrawn from the expandable structure 22 by drawing the inflation medium 128 into the syringe assembly 120′. The steerable catheter 10 can then be removed from the bone lesion 106 by straightening the steerable element 30 using the controller 18, or by simply allowing the steering element 30 to be straightened as the steerable catheter 10 is pulled out through the cannula 104, or by a combination of both. The physician may alternatively determine or confirm the volume of the bone lesion 106 by measuring the amount of inflation medium 128 withdrawn from the expandable structure 22.

In the embodiment pictured in FIG. 9. after cavity formation and deflation of the expandable member 22, the physician may slightly withdraw the steerable element within the bone lesion 106 and inject flowable bone cement 140 through port 40 into lumen 24. The flowable bone cement will travel from lumen 24 into lumen 34 of the steerable element 30 before it exits the steerable catheter 10 through the distal opening 39. Flowable bone cement 140 can be injected into the bone lesion 106 until the flowable bone cement 140 completely fills the bone lesion 106. The flowable bone cement 140 can be introduced into the steerable catheter by any type of material delivery system, including a syringe as pictured.

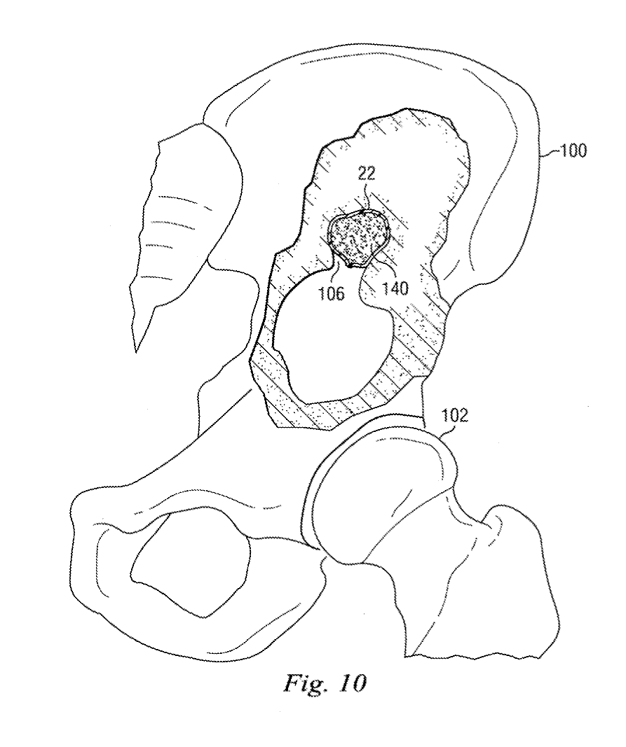

In some embodiments, the expandable member 22 can be left within the bone lesion 106 after cavity formation, and flowable bone cement 140 can be injected into the expandable structure 22 until the expandable structure 22 expands to occupy the entire bone lesion 106. FIG. 10 shows the expandable structure 22 left within the bone lesion 106 after being filled with flowable bone cement 140. Upon hardening, the bone cement 140 provides structural support for the bone of the ilium 100 surrounding the bone lesion 106, thereby substantially restoring the structural integrity of the hip.

Note that while the usage of the steerable catheter 10 is described for exemplary purposes as a sequential process involving the insertion of the catheter 10 into the bone lesion, articulation of the steerable element 30, aspiration or suction of the bone lesion, and inflation of the expandable member 22, any number and sequence of placement and positioning steps can be performed. For example, in one embodiment, the steerable catheter 10 could be placed in the bone lesion, the steerable element 30 could be articulated, the steerable catheter 10 could be moved further into the bone lesion, and the steerable element 30 could be articulated again before inflation of the expandable member 22. Alternatively, the steerable element 30 could be articulated repeatedly after inflation of the expandable member 22 to better position the expandable member 22 and thereby more precisely fill the bone lesion. In various other embodiments, the steerable catheter 10 could be moved further inward or outward relative to the cannula 104 concurrently with articulation of the steerable element 30.

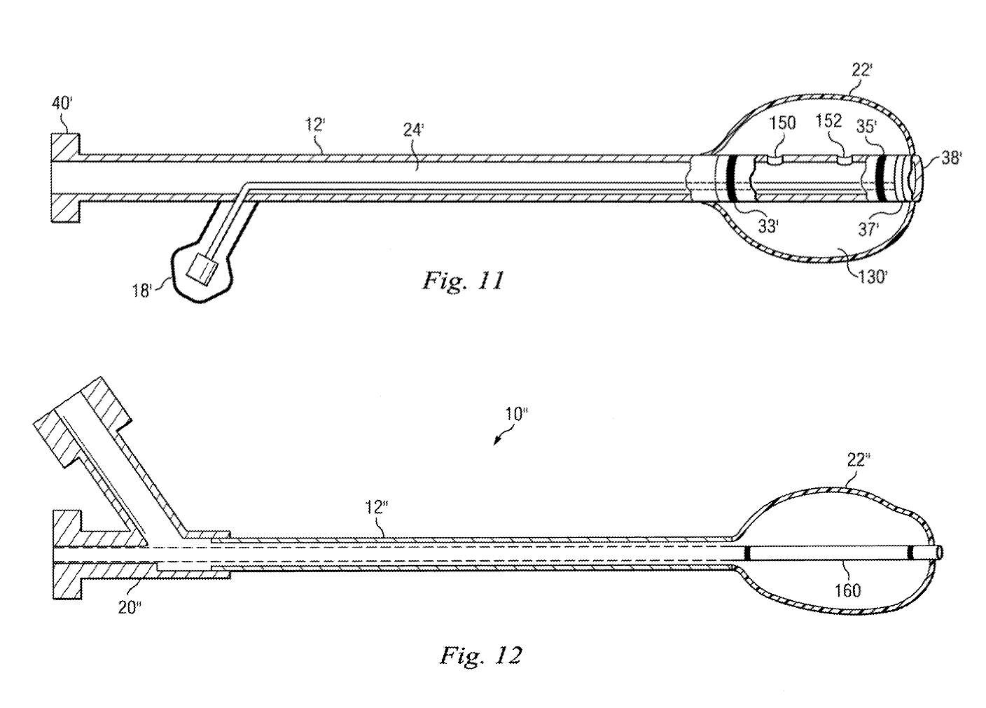

In the embodiment pictured in FIG. 1 a, the steerable catheter 10 possesses two ports 40 and 44 and two central lumens 24 and 23. In other embodiments, the steerable catheter 10′ may possess only one port 40′ and only one lumen 24′. For example, FIG. 11 depicts a second embodiment of a steerable catheter 10′ including a single port 40′ and a single central lumen 24′ of the shaft 12′. The lumen 24′ of the shaft 12′ is contiguous with and fluidly connected to the lumen 34′ of the steerable element 30′. The steerable element 30′ includes apertures 150, 152 disposed proximal to distal end 38′. In this embodiment, the apertures 150, 152 are disposed between radiopaque markers 33′ and 35′. Other embodiments may possess any number of apertures disposed along the length of steerable element 30′. The port 40′ provides for the releasable connection of the steerable catheter 10′ to a source of flowable material. The lumen 42′ of the port 40′ is fluidly connected to the lumen 24′ of the shaft 12 such that material can flow from a source, through the port 40′ into the port lumen 42′, into the lumen 24′ of the shaft 12, into the lumen 34′ of the steerable element 30′, and out the apertures 150, 152 of the steerable element 30′ into the lumen 130′ of the expandable structure 22′. In the embodiment pictured in FIG. 11, the steerable element 30′ includes a closed distal end 38′, thereby facilitating inflation of the expandable structure 22′. The steerable catheter 10′ also includes a steering mechanism 16′ that is housed within the lumen 24′. In some embodiments, the steering mechanism 16′ may be encased in flexible tubing such as polyamide tubing and be housed within the wall of the shaft 12′.

In another embodiment of the present invention, the steerable catheter may not have a steering mechanism 16. Instead, the steerable catheter is shaped and configured to function in cooperation with a separate steerable guide catheter having a lumen sized and configured to slidably accommodate the steerable catheter. For example, FIG. 12 illustrates a third embodiment of the steerable catheter 10″ including a shaft 12″, a connector 20″, an expandable structure 22″, and a distal portion 160. The expandable structure 22″ is configured to surround the distal portion 160. A physician may position the steerable guide catheter within a bone lesion, and then slide the steerable catheter 10″ through the lumen of the steerable guide catheter until the distal portion 160 of the steerable catheter 10″ emerges from the distal end of the steerable guide catheter and enters the bone lesion. Once the distal portion 160 exits the steerable guide catheter, the physician may assess the volume of the bone lesion as described above.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made. What is claimed is:

1. A method of assessing the volume of a lesion in a bone, the method comprising:
inserting a steerable catheter comprising an expandable structure, a suction member, and a steerable element into the bone along a longitudinal axis;
steering the steerable element away from the longitudinal axis toward the lesion;
removing cellular matter from the lesion using the suction member;
inflating the expandable structure with inflation medium to create a cavity defining the boundary of the lesion; and
measuring the volume of inflation medium in the expandable structure, thereby determining the volume of the cavity.

2. The method of claim 1, wherein the expandable structure is a compliant balloon.

3. The method of claim 1, wherein the inflation medium comprises sterile saline.

4. The method of claim 1, wherein the inflation medium comprises radiographic contrast.

5. The method of claim 1, wherein the expandable structure includes heating elements on its outer surface and including the step of heating the heating elements after inflation of the expandable structure such that a shell of cancellous bone is formed around the expandable structure.

6. The method of claim 1, wherein measuring the volume of inflation medium comprises measuring the volume of inflation medium after it is delivered to the expandable structure.

7. The method of claim 1, further comprising the step of deflating the expandable structure, and where measuring the volume of inflation medium comprises measuring the volume of inflation medium removed from the expandable structure to deflate the expandable structure.

8. The method of claim 1, wherein the method further comprises assessing the volume of the cavity by visually imaging the quantity of inflation medium in the expandable structure.

9. The method of claim 1, wherein the method further comprises filling the cavity with a material that sets to a hardened condition.

10. The method of claim 1, wherein the method further comprises leaving the expandable structure within the cavity after inflation and filling the expandable structure with a material that sets to a hardened condition.

11. The method of claim 1, wherein the steerable catheter further comprises:
an elongate shaft, wherein the expandable structure is coupled to the distal end of the elongate shaft and the steerable element is coupled to the expandable structure; and
a controller disposed on a proximal end of the elongate shaft, wherein the controller steers the steerable element.

12. The method of claim 11, wherein the controller comprises a rotatable element for articulating the steerable element.

13. The method of claim 1, wherein the steerable element extends at least partially into the expandable structure.

14. The method of claim 12, wherein a distal end of the steerable element is coupled to a distal end of the expandable structure.

15. The method of claim 1, wherein the expandable structure expands proximal to the steerable element.

16. The method of claim 1, wherein the expandable structure expands around the steerable element.

17. A method of assessing the volume of a lesion in a bone, the method comprising:
inserting a steerable catheter comprising an expandable structure, a suction member, and a steerable element into the bone, the steerable element including radiopaque markers;
steering the steerable element away from the longitudinal axis toward the lesion;
removing cellular matter from the lesion using the suction member;
inflating the expandable structure with inflation medium to create a cavity defining the boundary of the lesion; and
imaging the cavity while the expandable structure is inflated;
visualizing the radiopaque markers in the imaged cavity; and
measuring the volume of inflation medium in the expandable structure, thereby determining the volume of the cavity.

18. The method of claim 17, wherein the expandable structure is a compliant balloon.

19. The method of claim 17, wherein the expandable member includes integrated radiopaque markers.

20. The method of claim 17, wherein the expandable structure includes heating elements on its outer surface and including the step of heating the heating elements after inflation of the expandable structure such that a shell of cancellous bone is formed around the expandable structure.

21. The method of claim 17, wherein measuring the volume of inflation medium comprises measuring the volume of inflation medium after it is delivered to the expandable structure.

22. The method of claim 17, further comprising the step of deflating the expandable structure, and where measuring the volume of inflation medium comprises measuring the volume of inflation medium removed from the expandable structure to deflate the expandable structure.

23. The method of claim 17, wherein the method further comprises assessing the volume of the cavity by visually imaging the quantity of inflation medium in the expandable structure.

24. The method of claim 17, wherein the method further comprises estimating the volume of the cavity by visually imaging the positional relationship of the radiopaque markers and measuring their relative separation.

25. The method of claim 17, wherein the method further comprises filling the cavity with a material that sets to a hardened condition.

26. The method of claim 17, wherein the method further comprises leaving the expandable structure within the cavity after inflation and filling the expandable structure with a material that sets to a hardened condition.

27. The method of claim 17, wherein the steerable catheter further comprises:
an elongate shaft, wherein the expandable structure is coupled to the distal end of the elongate shaft and the steerable element is coupled to the expandable structure; and
a controller disposed on a proximal end of the elongate shaft, wherein the controller steers the steerable element.

28. A method of assessing the volume of a lesion in a bone, the method comprising:
inserting a steerable catheter comprising an expandable structure, a suction member, and a steerable element into the bone, the steerable element being connected to the expandable structure;
steering the steerable element away from the longitudinal axis toward the lesion;
removing cellular matter from the lesion using the suction member;
inflating the expandable structure with inflation medium to create a cavity defining the boundary of the lesion; and
articulating the steerable element to change a configuration of the expandable structure;
imaging the cavity while the expandable structure is inflated; and
measuring the volume of inflation medium in the expandable structure, thereby determining the volume of the lesion.

29. The method of claim 27, wherein articulating the steerable element to change a configuration of the expandable structure occurs before inflation of the expandable structure.

30. The method of claim 27, wherein articulating the steerable element to change a configuration of the expandable structure occurs during inflation of the expandable structure.

31. The method of claim 27, wherein the steerable element extends at least partially into the expandable structure and the expandable structure expands around the steerable element.
